(19) United States
(12) Patent Application Publication
(10) Pub. No.: US 2011/0190899 A1
(43) Pub. Date: Aug. 4, 2011

(54) PATIENT-SPECIFIC AUGMENTS

(75) Inventors: Andrew L. Pierce, Warsaw, IN (US); Robert Metzger, Wakarusa, IN (US)

(73) Assignee: BIOMET MANUFACTURING CORP., Warsaw, IN (US)

(21) Appl. No.: 13/047,924

(22) Filed: Mar. 15, 2011

Related U.S. Application Data
(63) Continuation-in-part of application No. 12/973,214, filed on Dec. 20, 2010.
Application No. 12/973,214 is a continuation-in-part of application No. 12/955,361, filed on Nov. 29, 2010.
Application No. 12/955,361 is a continuation-in-part of application No. 12/938,905, filed on Nov. 3, 2010.
Application No. 12/938,905 is a continuation-in-part of application No. 12/938,913, filed on Nov. 3, 2010.
Application No. 12/938,905 is a continuation-in-part of application No. 12/893,306, filed on Sep. 29, 2010.
Application No. 12/938,913 is a continuation-in-part of application No. 12/893,306, filed on Sep. 29, 2010.
Application No. 12/893,306 is a continuation-in-part of application No. 12/888,005, filed on Sep. 22, 2010.
Application No. 12/888,005 is a continuation-in-part of application No. 12/714,023, filed on Feb. 26, 2010.
Application No. 12/714,023 is a continuation-in-part of application No. 12/571,969, filed on Oct. 1, 2009.
Application No. 12/571,969 is a continuation-in-part of application No. 12/486,992, filed on Jun. 18, 2009.
Application No. 12/486,992 is a continuation-in-part of application No. 12/389,901, filed on Feb. 20, 2009.
Application No. 12/389,901 is a continuation-in-part of application No. 12/211,407, filed on Sep. 16, 2008.
Application No. 12/211,407 is a continuation-in-part of application No. 12/039,849, filed on Feb. 29, 2008.
Application No. 12/039,849 is a continuation-in-part of application No. 11/756,057, filed on May 31, 2007.
Application No. 12/039,849 is a continuation-in-part of application No. 11/971,390, filed on Jan. 9, 2008.
Application No. 11/971,390 is a continuation-in-part of application No. 11/363,548, filed on Feb. 27, 2006, now Pat. No. 7,780,672.
Application No. 12/039,849 is a continuation-in-part of application No. 12/025,414, filed on Feb. 4, 2008.
Application No. 12/025,414 is a continuation-in-part of application No. 12/872,663, filed on Aug. 31, 2010.
Application No. 12/872,663 is a continuation-in-part of application No. 12/483,807, filed on Jun. 12, 2009.
Application No. 12/483,807 is a continuation-in-part of application No. 12/371,096, filed on Feb. 13, 2009.
Application No. 12/371,096 is a continuation-in-part of application No. 12/103,824, filed on Apr. 16, 2008.
Application No. 12/103,824 is a continuation-in-part of application No. 12/103,834, filed on Apr. 16, 2008.
Application No. 12/103,834 is a continuation-in-part of application No. 12/978,069, filed on Dec. 23, 2010.
Application No. 12/978,069 is a continuation-in-part of application No. 12/973,214, filed on Dec. 20, 2010.
Application No. 12/973,214 is a continuation-in-part of application No. 13/041,469, filed on Mar. 7, 2011.
Application No. 13/041,469 is a continuation-in-part of application No. 13/041,495, filed on Mar. 7, 2011.
Application No. 13/041,495 is a continuation-in-part of application No. 13/041,665, filed on Mar. 7, 2011.
Application No. 13/041,665 is a continuation-in-part of application No. 13/041,883, filed on Mar. 7, 2011.
(60) Provisional application No. 61/446,660, filed on Feb. 25, 2011.
(60) Provisional application No. 60/953,620, filed on Aug. 2, 2007.
(60) Provisional application No. 60/947,813, filed on Jul. 3, 2007.
(60) Provisional application No. 60/911,297, filed on Apr. 12, 2007.
(60) Provisional application No. 60/892,349, filed on Mar. 1, 2007.
(60) Provisional application No. 60/812,694, filed on Jun. 9, 2006.
(60) Provisional application No. 60/953,637, filed on Aug. 2, 2007.
(60) Provisional application No. 61/310,752, filed on Mar. 5, 2010.
(60) Provisional application No. 60/912,178, filed on Apr. 17, 2007.
(60) Provisional application No. 60/912,178, filed on Apr. 17, 2007.

(51) Int. Cl. A61F 2/38; A61F 2/30

(52) U.S. Cl. 623/20.32; 623/18.11

(57) ABSTRACT

A patient-specific augment can be attached to an implant component for a bone of a joint of a patient. The patient-specific augment has first and second surfaces. The first surface is a three-dimensional patient-specific surface that closely matches and can mate to a substantially unaltered and unresected surface of a bone defect of the specific patient only in one position. The second surface is designed to engage a non-custom surface of the implant.

### CROSS-REFERENCE TO RELATED APPLICATIONS

This application claims the benefit of U.S. Provisional Application No. 61/446,660, filed on Feb. 25, 2011.

This application is a continuation-in-part of U.S. application Ser. Nos. 13,041,469, 13/041,495, 13/041,665 and 13/041,883, each filed on Mar. 7, 2011, each of which is a continuation-in-part of U.S. application Ser. No. 12/978,069 filed Dec. 23, 2010, which is a continuation-in-part of U.S. application Ser. No. 12/973,214, filed Dec. 20, 2010, which is a continuation-in-part of U.S. application Ser. No. 12/955,361 filed Nov. 29, 2010, which a continuation-in-part of U.S. application Ser. Nos. 12/938,905 and 12/938,913, both filed Nov. 3, 2010, each of which is a continuation-in-part of U.S. application Ser. No. 12/893,306, filed Sep. 29, 2010, which is a continuation-in-part of U.S. application Ser. No. 12/888,005, filed Sep. 22, 2010, which is a continuation-in-part of U.S. application Ser. No. 12/714,023, filed Feb. 26, 2010, which is a continuation-in-part of U.S. application Ser. No. 12/571,969, filed Oct. 1, 2009, which is a continuation-in-part of U.S. application Ser. No. 12/486,992, filed Jun. 18, 2009, and is a continuation-in-part of U.S. application Ser. No. 12/389,901, filed Feb. 20, 2009, which is a continuation-in-part of U.S. application Ser. No. 12/211,407, filed Sep. 16, 2008, which is a continuation-in-part of U.S. application Ser. No. 12/039,849, filed Feb. 29, 2008, which: (1) claims the benefit of U.S. Provisional Application No. 60/953,620, filed on Aug. 2, 2007, U.S. Provisional Application No. 60/947,813, filed on Jul. 3, 2007, U.S. Provisional Application No. 60/911,297, filed on Apr. 12, 2007, and U.S. Provisional Application No. 60/892,349, filed on Mar. 1, 2007; (2) is a continuation-in-part U.S. application Ser. No. 11/756,057, filed on May 31, 2007, which claims the benefit of U.S. Provisional Application No. 60/812,694, filed on Jun. 9, 2006; (3) is a continuation-in-part of U.S. application Ser. No. 11/971,390, filed on Jan. 9, 2008, which is a continuation-in-part of U.S. application Ser. No. 11/363,548, filed on Feb. 27, 2006; and (4) is a continuation-in-part of U.S. application Ser. No. 12/025,414, filed on Feb. 4, 2008, which claims the benefit of U.S. Provisional Application No. 60/953,637, filed on Aug. 2, 2007.

This application is continuation-in-part of U.S. application Ser. No. 12/872,663, filed on Aug. 31, 2010, which claims the benefit of U.S. Provisional Application No. 61/310,752 filed on Mar. 5, 2010.

This application is a continuation-in-part of U.S. application Ser. No. 12/483,807, filed on Jun. 12, 2009, which is a continuation-in-part of U.S. application Ser. No. 12/371,096, filed on Feb. 13, 2009, which is a continuation-in-part of U.S. application Ser. No. 12/103,824, filed on Apr. 16, 2008, which claims the benefit of U.S. Provisional Application No. 60/912,178, filed on Apr. 17, 2007.

This application is also a continuation-in-part of U.S. application Ser. No. 12/103,834, filed on Apr. 16, 2008, which claims the benefit of U.S. Provisional Application No. 60/912,178, filed on Apr. 17, 2007.

The disclosures of the above applications are incorporated herein by reference.

### INTRODUCTION

The present teachings provide various patient-specific augments used with implant components for the knee joint.

### SUMMARY

The present teachings provide a patient-specific augment that can be attached to an implant component for a bone of a joint of a patient. The patient-specific augment has first and second surfaces. The first surface is a three-dimensional patient-specific surface that closely matches and can mate to a substantially unaltered and unresected surface of a bone defect of the specific patient only in one position. The second surface is designed to engage a non-custom surface of the implant.

The augment can include a patient-specific peripheral surface between the first and second surfaces. The peripheral surface can have a patient-specific variable depth and/or a patient-specific variable radial dimension. In some embodiments, the augment can have a patient-specific step discontinuity in depth.

In some embodiments, the augment can be a femoral augment for a femoral knee component. In some embodiments, the augment can be a tibial augment for a tibial knee component. In some embodiments, the augment can be in the form of a sleeve with a generally tapered or cylindrical surface. In some embodiments, the augment can be generally block or plate-like.

The present teachings also include a method of preparing a knee joint for an implant component. The method includes preparing a bone end of the knee joint of the patient with standard resections configured to match a non-custom bone-engaging surface of an implant component without resecting a three-dimensional surface of a defect in the bone. A first implant-engaging surface of a patient-specific augment is attached to a portion of the bone-engaging surface of the implant. A second patient-specific three-dimensional surface of the augment is nestingly mated to the three-dimensional surface of the defect, and the implant is attached to the bone. According to the method, the patient-specific augment is designed during a preoperative plan for the specific patient. A three-dimensional image of the bone with the defect is reconstructed from a medical scan of the bone during a preoperative plan. An implant component is selected for the patient, and the patient-specific augment is manufactured from the three-dimensional image of the bone of the specific patient and the implant component.

Further areas of applicability of the present teachings will become apparent from the description provided hereinafter. It should be understood that the description and specific examples are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1 is an environmental perspective view of a prior art knee replacement joint in which a location of an exemplary tibial bone defect is illustrated;

FIG. 2 is an environmental perspective view of a prior art femoral implant in which exemplary femoral bone defects are illustrated;

FIG. 3 is a perspective view of a prior art femoral implant illustrating non-custom distal and posterior femoral augments;

FIG. 4 is a perspective view of a prior art tibial implant illustrating a non-custom augment;

FIG. 4A illustrates a distal femoral bone on which representative standard cuts are shown for receiving an illustrated standard non-custom femoral implant;

FIG. 5 is a perspective view of a femoral implant illustrating a patient-specific distal augment according to the present teachings;

FIG. 6 is a perspective view of an exemplary patient-specific distal femoral augment according to the present teachings;

FIG. 7 is a perspective view of an exemplary patient-specific posterior femoral augment according to the present teachings;

FIG. 8 is a perspective view of an exemplary patient-specific tibial tray augment according to the present teachings;

FIG. 9 is an environmental plan view of an exemplary patient-specific tibial augment according to the present teachings;

FIG. 10 is an environmental elevated view of the patient-specific tibial augment of FIG. 9;

FIG. 11 is a perspective view of the patient-specific tibial augment of FIG. 10; and

FIG. 12 is a perspective view of an exemplary patient-specific femoral augment according to the present teachings.

### DESCRIPTION OF VARIOUS ASPECTS

The following description is merely exemplary in nature and is in no way intended to limit the present teachings, applications, or uses.

The present teachings generally provide various patient-specific augments for knee implants. The patient-specific augments are implants that can be used either with conventional femoral or tibial implant components. The patient-specific augments can be designed using computer-assisted image methods based on three-dimensional images of the patient's knee anatomy reconstructed from MRI, CT, ultrasound, X-ray or other three- or two-dimensional medical scans of the patient's anatomy. Various CAD programs and/or software can be utilized for three-dimensional image reconstruction, such as software commercially available, for example, by Materialise USA, Ann Arbor, Mich.

According to the present teachings, the augments are customized to the anatomy of a specific patient to correct defects, irregularities and other deformities without having to sacrifice any additional amount of bone, as required by standard, non custom augments.

The patient-specific augments are generally formed using computer modeling based on the patient's three-dimensional (3-D) anatomic image and have an engagement surface that is made to conformingly contact and match a three-dimensional image of the patient's bone surface, by the computer methods discussed above. Various preoperative planning procedures and patient-specific instruments are disclosed in commonly assigned and co-pending U.S. patent application Ser. No. 11/756,057, filed on May 31, 2007; U.S. patent application Ser. No. 12/211,407, filed Sep. 16, 2008; U.S. patent application Ser. No. 11/971,390, filed on Jan. 9, 2008, U.S. patent application Ser. No. 11/363,548, filed on Feb. 27, 2006; and U.S. patent application Ser. No. 12/025,414, filed Feb. 4, 2008. The disclosures of the above applications are incorporated herein by reference.

In the preoperative planning stage for a joint replacement or revision procedure, an MRI scan or a series of CT or other medical scans of the relevant anatomy of the patient, such as, for example, the bones (with or without articular cartilage) of the joint to be reconstructed, can be performed at a medical facility or doctor's office. The scan data obtained can be sent to a manufacturer. The scan data can be used to construct a three-dimensional image of the joint and provide an initial fitting in a computer file form or other computer representation.

The outcome of the initial fitting is an initial surgical plan that can be printed or provided in electronic form with corresponding viewing software. The initial surgical plan can be surgeon-specific, when using surgeon-specific alignment protocols. The initial surgical plan, in a computer file form associated with interactive software, can be sent to the surgeon, or other medical practitioner, for review. The surgeon can incrementally manipulate the position of images of the augment components and the implant components in an interactive image of the joint. Additionally, the surgeon can select or modify resection planes, types of augments, implants and orientations of augment and/or implant insertion. After the surgeon modifies and/or approves the surgical plan, the surgeon can send the final, approved plan to the manufacturer.

The present teachings provide patient-specific or custom implantable components or augments for use with femoral or tibial knee implants. Non-custom augments can be, for example, in the form of block augments, flat augments, conical, tapered and sleeve augments for the tibial and femoral implant components for augment corresponding areas of the patient's knee joint anatomy in connection with knee arthroplasty. The patient-specific augments of the present teachings have three-dimensional surfaces designed to closely conform to complementary bone surfaces of the patient's joint. Accordingly, the patient-specific augments of the present teachings deviate from the standard geometry of non-custom block, flat, conical, tapered and sleeve augments.

Each patient-specific augment generally includes a patient-specific surface mirroring and nestingly engaging a portion of the patient's anatomy and another surface that engages and mates to a corresponding (different) implant component in the manner of non custom augments. In this respect, the patient-specific augment can be designed for a particular patient to correct a defect while minimizing sacrificed bone and yet be used with an off-the-shelf non-custom implant component of a size appropriate for the patient.

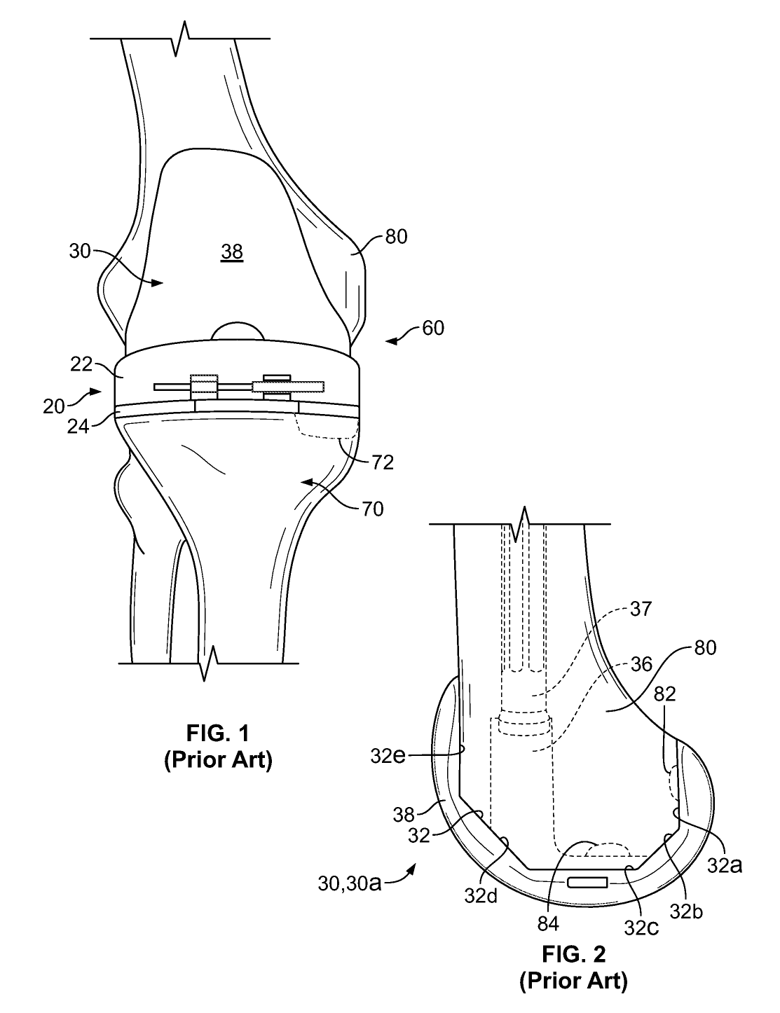

Referring to FIG. 1, a knee joint 60 between a tibia 70 and a distal femur 80 is illustrated. The knee joint 60 is shown with a total knee replacement including a femoral implant 30 and a tibial implant 20 with a bearing 22 and a tibial tray 24. An exemplary tibial defect 72, drawn schematically only, is marked in phantom lines to indicate a site for a possible tibial augment. The various standard size non-custom femoral implants illustrated herein (30a, 30b, 30c) are collectively referenced with the numeral 30.

Referring to FIG. 2, the distal femur 80 of a knee joint is shown with an exemplary (prior art) posterior stabilized distal femoral implant 30a including a boss 36 for a femoral stem 37. The femoral implant 30a has a bone-engaging surface 32 with five internal flat/planar surfaces or flanges 32a, 32b, 32c, 32d, 32e corresponding to five resected planar surfaces of the distal femur 80. In this example, 32a is a planar surface corresponding to a posterior resection, 32c is a planar surface corresponding to a distal resection, 32e is a planar surface corresponding to an anterior resection, and surfaces 32b and 32d are intermediate posterior and anterior chamfer surfaces respectively. Exemplary femoral defects, drawn schematically only, for possible femoral augments are illustrated at 82 (posterior) and 84 (distal). The bone-engaging surface 32 is opposite to the outer articulating surface 38 that faces the patient's joint.

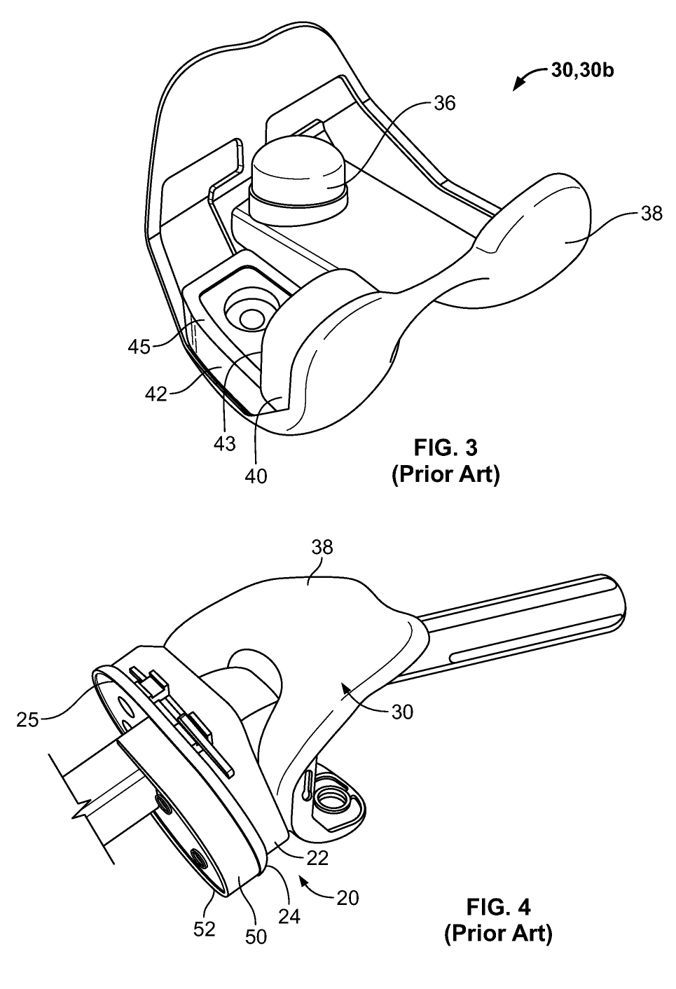

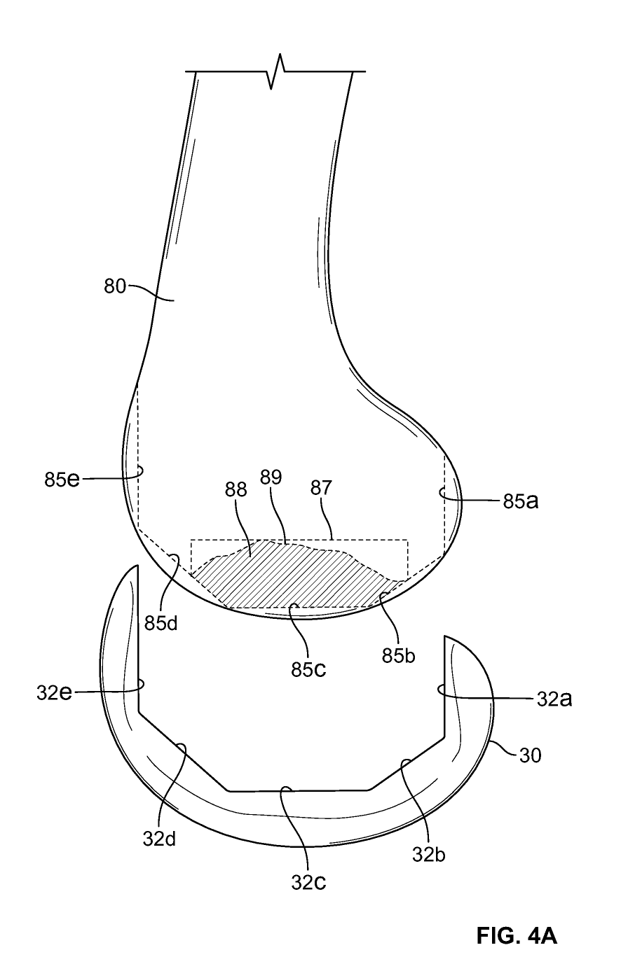

Referring to FIG. 3, another exemplary (prior-art) femoral implant 30b with an exemplary non-custom posterior femoral augment 40 and an exemplary non-custom distal femoral augment 42 is illustrated. As illustrated, regardless of the shape of the defect and geometry of the bone surface, non-custom femoral augments 40, 42 require a larger portion of the bone to be removed to accommodate the standard dimensions, sizes and shapes of the non-custom augments regardless of the shape of the defect and associated patient's anatomy. In other words, the corresponding bone-engaging surfaces 43, 45 of the non custom augments 40, 42 are substantially flat or planar requiring planar cuts to remove the defect instead of conforming to the shape of the defect to correct the defect with a relative continuous or seamless interface. Similarly, and referring to FIG. 4, a non-custom tibial augment 50 is illustrated under one side of the outer surface 25 of the tibial tray 24. The outer surface 25 of the tibial tray 24 is the surface that faces away from the joint. The non-custom tibial augment 50 has a substantially planar or flat bone-engagement surface 52 that requires bone removal typically in excess to the size of a defect and/or independently of the associated bone geometry.

As an example of the procedure used with prior art, non-custom augments, FIG. 4A illustrates a distal femur 80 that can be resected along five planar surfaces, specifically a posterior resection 85a, a posterior chamfer resection 85b, a distal resection 85c, an anterior chamfer resection 85d and an anterior resection 86e, for engaging the corresponding five inner planar surfaces 32a, 32b, 32c, 32d and 32e of a standard femoral implant 30. If the distal femur 80 includes a bone defect 88, a volume bigger than the defect 88 is generally removed along a new resection boundary 87 of planar surfaces to accommodate a prior art non-custom, block-type augment. In contrast, the method of the present teachings utilizes the standard resections outside the defect 88 and provides an augment that will be bounded by a substantially unaltered and unresected inner boundary 89 of the defect 88 and planar surfaces that correspond to portions of the standard resections (i.e., portions of 85d, 85c and 85b in FIG. 4A) for engaging corresponding portions of the inner planar surfaces of the femoral implant 30 (32d, 32c, and 32b). The method of the present teachins results in retaining more healthy host bone as opposed to simply cutting out the defect to crest planar surfaces. The bone defect 88 can be a void, a recess or an area of weakened, diseased or otherwise defective bone.

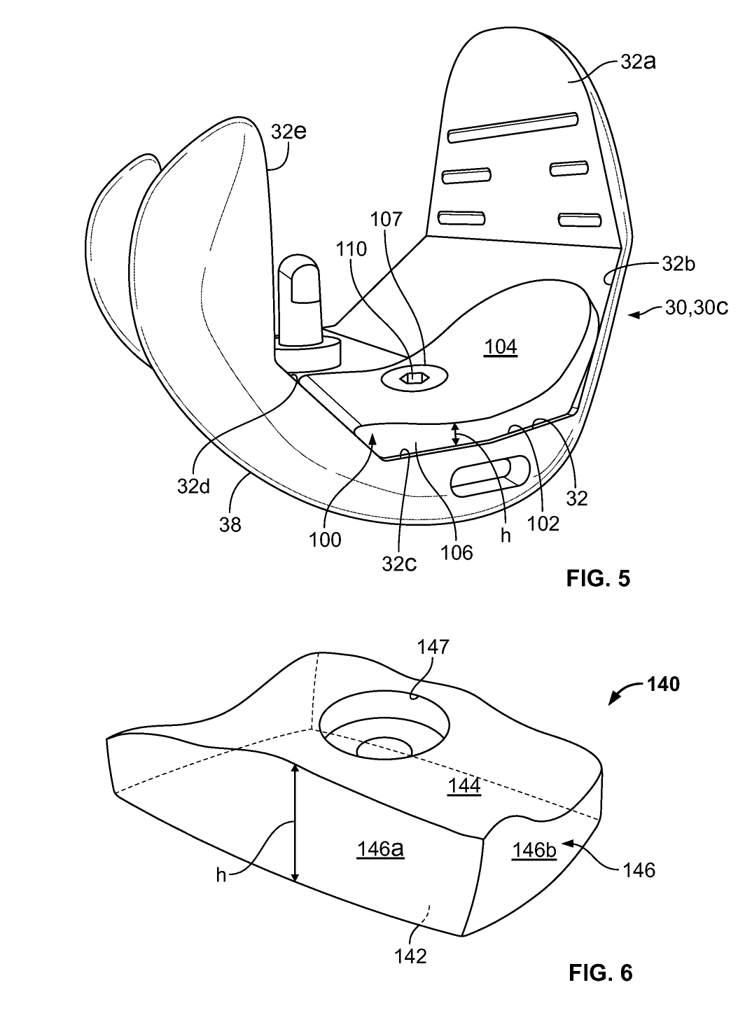

FIGS. 5-12 illustrate various exemplary patient-specific augments designed according to the present teachings. FIGS. 5-7 and 12 illustrate various patient-specific femoral augments. FIGS. 8-11 illustrate various patient-specific tibial augments. The various augments illustrated in FIGS. 5-8 replace flat or planar non-custom augments. The augments illustrated in FIGS. 5-8 are patient-specific and have bone-engagement surfaces that are designed to mate with the patient's bone anatomy, including a substantially unaltered and unresected surface of a bone defect of the patient, and are generally three-dimensional, curved, non flat surfaces. FIGS. 9-12 illustrate various patient-specific augments that replace non-custom conical, tapered or cylindrical augments and have patient-specific bone engagement surfaces with three-dimensional surfaces corresponding to the patient's anatomy.

The various augments can be made of biocompatible materials, including metals, titanium alloys, porous metals or other material with porous coatings for bone in-growth, including, for example, porous titanium, such as Regerenex®, commercially available from Biomet Manufacturing Corp, Warsaw, Ind.

More specifically, FIG. 5 illustrates an exemplary patient-specific femoral augment 100 for a non-custom knee femoral implant 30c. The femoral augment 100 has an implant-engaging multi-planar surface 102 designed to mateably engage, for example, portions of the standard inner planar surfaces 32d (anterior-distal), 32c (distal) and 32b (distal-posterior) of the femoral implant 30c. In some embodiments, the femoral implant 30c can be designed with an outer surface 38 that is patient-specific and matches, for example, the anatomy of a healthy articular surface of the femur of the patient. In either case, the patient-specific femoral augment 100 can engage any standard inner planar surfaces of a femoral implant 30 that is designed to be fitted on the distal femur of a patient using standard cuts, as discussed above.

The femoral augment 100 has a bone-engaging surface 104 generally opposite to an implant-engaging surface 102 and a three-dimensional peripheral bone-engaging surface 106. In this respect, the femoral augment 100 can be bounded by opposing surfaces 104, 102 and peripheral surface 106 that joins the opposing surfaces 102, 104. The femoral augment 100 can include a fixation aperture 107 for a fixation member 110, such as a fastener or set screw for coupling the femoral augment 100 to the femoral implant 30c. The femoral augment can also be cemented to the femoral implant 30c. The bone-engaging surface 104 is a patient-specific surface designed according to the preoperative plan for the patient based on a three-dimensional reconstructed image of the patient's anatomy, including a substantially unaltered and unresected surface of a bone defect of the patient, using medical scans, as discussed above. The patient-specific surface 104 is generally a curved three-dimensional surface designed to closely and nestingly mate in mirror-image fashion with a complementary surface of the specific patient's bone, including any defects, asymmetries or other irregularities for correcting such irregularities. For example, the bone-engaging surface 104 of the femoral augment 100 can include convex, concave, planar or other portions with generally continuous or piece-wise variable geometry and/or curvature. The peripheral surface 106, to the extent that it engages the patient's anatomy, can also be designed to be patient-specific or have patient-specific portions based on the preoperative plan. The thickness or depth h of the augment 100 is patient-specific and can also be variable or non-uniform, as shown in FIG. 5.

Referring to FIG. 6, another patient-specific femoral augment 140 is illustrated. The patient-specific femoral augment 140 can be, for example, a distal or posterior or anterior or other type of femoral augment having a patient-specific bone-engaging surface 144, designed according to a preoperative plan for the patient to match a substantially unaltered and unresected surface of a bone defect of the patient, and an opposite, implant-engaging surface 142 that can be planar or piece-wise planar to mate with corresponding inner surface or surfaces (one or more portions of 32a, 32b, 32c, 32d, and 32e) of a femoral implant 30 (see FIG. 5. for example). The femoral augment 140 can include a fixation aperture 147. The depth h of the augment 140, i.e., the distance between the patient-specific bone-engaging surface 144 and the implant-engaging surface 142, can be patient-specific and non-uniform or variable over the area of the augment 140. The augment 140 can include a three-dimensional peripheral bone-engaging surface 146, which can be patient-specific, or have patient-specific portions (continuous or spaced apart), as dictated by the geometry of the defect of the specific patient, an image of which is reconstructed during the preoperative plan for the patient and nestingly matched by the augment 140. The peripheral bone-engaging surface 146 can be a continuous surface, or, alternatively, can include a number of side surfaces, such as side surfaces 146a and 146b and their opposites, for four-sided augments 140. It is contemplated that irregularly shaped augments with a different number of sides or a continuous peripheral bone-engaging surface, in addition to the implant-engaging surface 142, can be used depending on the anatomy of the specific patient.

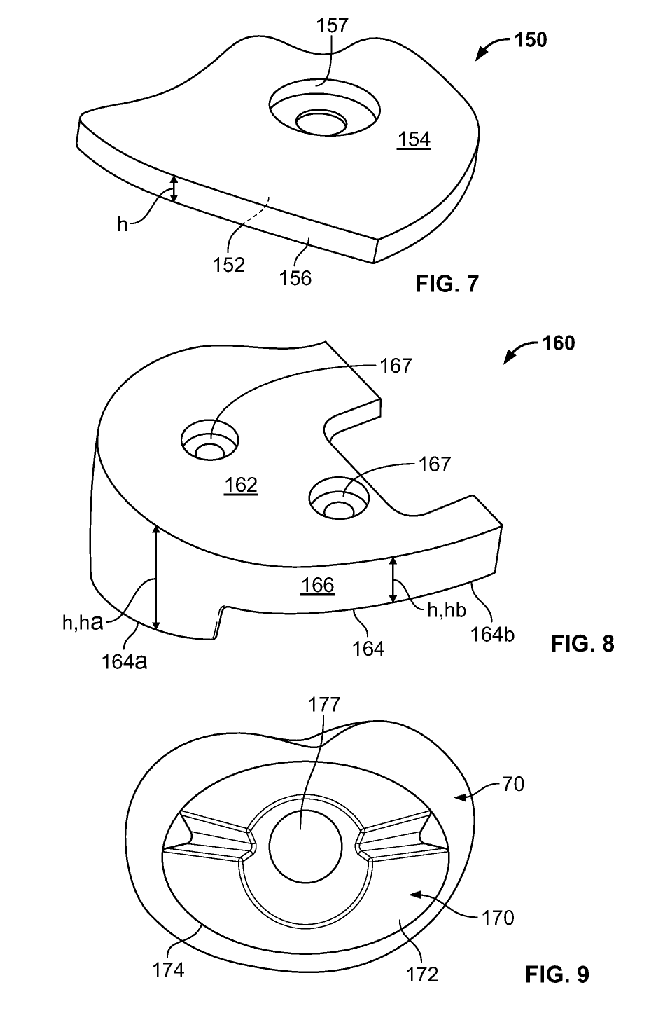

Referring to FIG. 7, another patient-specific femoral augment 150 is illustrated. The patient-specific femoral augment 150 can be, for example, a patient-specific posterior femoral replacing for example, a non-custom augment similar to the augment 40 of FIG. 3, or other patient-specific augment replacing a non-custom block-type femoral augment. The femoral augment 150 has a bone-engaging surface 154 (designed according to a preoperative plan for the patient) and an opposite, implant-engaging surface 152 that can be planar or piece-wise planar to mate with corresponding inner surface or surfaces of a femoral implant 30, shown in FIG. 3. The femoral augment 150 can include a fixation aperture 157 for receiving a fastener or a set screw to secure the femoral augment 150 to the corresponding femoral implant. The femoral augment 150 can also be cemented on the femoral implant. The femoral augment 150 can include a three-dimensional peripheral bone-engaging surface 156 with a non-uniform profile, which can be patient-specific (or have patient-specific portions) and based on the preoperative plan for the patient. The depth h of the femoral augment 150, i.e., the distance between the patient-specific bone-engaging surface 154 and the implant-engaging surface 152, can be substantially constant, i.e., the femoral augment 150 can be substantially block or plate-like, although in some embodiments the depth h can be non-uniform or variable over the femoral augment 150.

Referring the FIG. 8, a patient-specific tibial augment 160 is illustrated. The tibial augment 160 has a bone-engaging surface 164, designed according a preoperative plan to nestingly match and mate with the anatomy of the patient, including a substantially unaltered and unresected surface of a bone defect of the patient, and an opposite, implant-engaging surface 162 that can be planar or piece-wise planar to mate with corresponding inner surface or surfaces of tibial implant components, such as a tibial tray 24, shown in FIG. 4. The tibial augment 160 can include a fixation aperture 167. The tibial augment 160 can include a three-dimensional peripheral bone-engaging surface 166 with a non-uniform profile, which can be patient-specific or have patient-specific portions, depending on the geometry and location of the defect that it replaces and based on the preoperative plan for the patient. The depth h of the tibial augment 160, i.e., the distance between the patient-specific bone-engaging surface 164 and the implant-engaging surface 162, can be patient-specific and variable over the tibial augment 160. In some embodiments, the depth h can include a patient-specific step discontinuity between the first and second bone-engaging portions 164a, 164b of the bone-engaging surface 164. Each bone-engaging portion 164a, 164b can have a corresponding depth ha, hb, as shown in FIG. 8, with a patient-specific step discontinuity in the values of ha and hb. Within each bone-engaging portion 164a, 164b, the corresponding depth can be substantially constant or continuously variable (with no additional discontinuities). In some embodiments, more than two discontinuous bone-engaging portions 164a, 164b may be present.

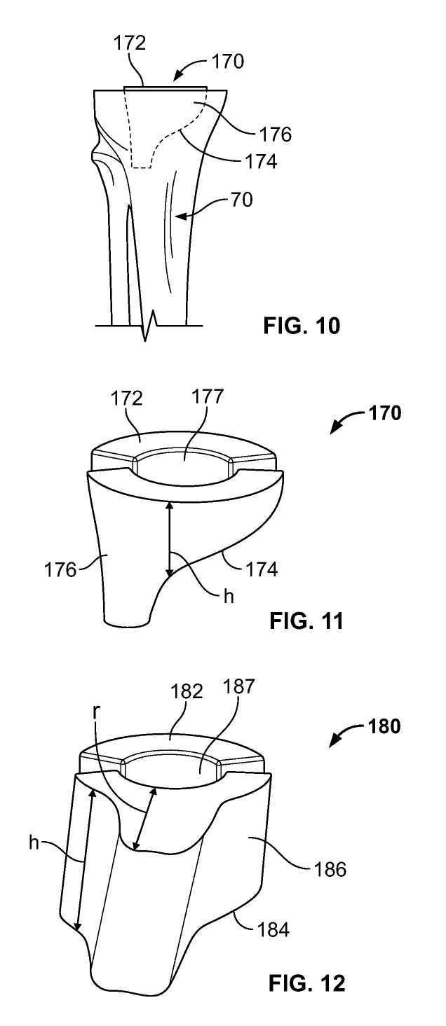

FIGS. 9-11 illustrate various views of a patient-specific augment 170 for a patient which would ordinarily receive a non-custom uniform conical or tapered augment. The patient-specific augment 170 can be in the form of a tapered sleeve and has a proximal, substantially planar implant-engaging surface 172 including a fixation formation 177. Although the patient-specific augment 170 is illustrated for the tibia in FIGS. 9 and 10, the augment 170 can be similarly designed as a patient-specific stem augment for the femoral boss 36 or femoral stem 37 of femoral implant 30, shown in FIG. 2, which can be received in a bore-shaped fixation formation 177 and secured with a taper to taper connection or with cement or other fastener. The patient-specific augment 170 can include a peripheral, three-dimensional, partially conical, tapered or cylindrical bone-engaging surface 176 with an irregular distal (for tibial augment) patient-specific bone-engaging surface 174, such that a depth h of the augment 170 is patient-specific and variable, or non-uniform, resulting in a patient-specific tapered-type augment of variable depth corresponding to the patient's bone defect and other characteristics. The peripheral bone-engaging surface 176 can also be patient-specific or have portions that are patient-specific, as determined during the preoperative plan for the patient.

Referring to FIG. 12, another patient-specific augment 180 for a tibial or femoral implant is illustrated. The patient-specific augment 180 can be in the form of a cylindrical or tapered sleeve and include a fixation formation 187, such as a bore for a stem or boss of a femoral or tibial implant. A taper fit connection (taper to taper) can be used for augments in the form of sleeves. The patient-specific augment 180 can include a first surface 182 and a second surface 184 opposite to the first surface 182. The first and second surfaces 182, 184 are bounded by a three-dimensional peripheral surface 186. As illustrated in FIG. 12, the first surface 182 is a substantially planar implant-engaging surface. The second surface 184 and the peripheral surface 186 are patient-specific and generally three-dimensional bone-engaging surfaces. In some embodiments, the depth h of the augment 180, i.e., the distance between the first and second surfaces 182, 184 can be constant, while in other embodiments the depth can be patient-specific and variable reflecting the patient's anatomy, including a substantially unaltered and unresected surface of a bone defect of the patient. Similarly, the radial dimension “r” at each depth can be patient-specific and variable reflecting the patient's anatomy, including a substantially unaltered and unresected surface of a bone defect of the patient. In this respect, the peripheral surface 186 can be a three-dimensional surface with variable curvature and can include, for example, concave and convex portions, steps or other discontinuities that are complementary and closely match the patient's anatomy, including a substantially unaltered and unresected surface of a bone defect of the patient, as determined during a preoperative plan for the patient.

The present teachings generally provide various augments to be used with different implant components for a joint, such as the knee. Each of the augments of the present teachings are patient-specific and are designed for the anatomic features, including a substantially unaltered and unresected surface of a bone defect of a specific patient, based on a preoperative plan for the patient in which a three-dimensional computer image of the patient's joint is constructed. Using patient-specific augments that closely conform to the patient's geometry helps correct defects and other irregularities with reduced or minimal bone removed, while non-custom joint implants and other joint replacement components can fit as planned for the specific patient. In this respect, the augments of the present teachings can be used with non-custom implants, such as non-custom femoral and tibial knee implants. It is contemplated, however that the augments of the present teachings can also be used with implants having patient-specific geometries in areas other than those engaging the augment. For example, the femoral implant 30 can be designed with an outer surface 38 that is patient-specific and matches, for example, the anatomy of a healthy articular surface of the femur of the patient.

The various augments can be attached to the corresponding implants with cement, screws, bolts, set screws or other fasteners, or received in counter recesses of the implant or secured with a taper fit connection (taper to taper) in corresponding bores of the implant, as discussed above.

The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the present teachings without departing from the essential scope thereof. One skilled in the art will readily recognize from such discussion, and from the accompanying drawings and claims, that various changes, modifications and variations can be made therein without departing from the spirit and scope of the present teachings as defined in the following claims.

What is claimed is:

1. An implantable device comprising:
a patient-specific augment attachable to an implant component for a bone of a joint of a patient, the augment having first and second surfaces, wherein the first surface is a three-dimensional patient-specific surface that closely matches and can mate to a substantially unaltered and unresected surface of a bone defect of the specific patient only in one position, and wherein the second surface is designed to engage a non-custom surface of the implant.

2. The implantable device of claim 1, wherein the augment includes a peripheral surface between the first and second surfaces, wherein the peripheral surface is a three-dimensional surface that closely matches and can mate to a complementary surface of the bone of the specific patient.

3. The implantable device of claim 2, wherein the augment has a patient-specific and variable depth between the first and second surfaces.

4. The implantable device of claim 3, wherein the depth of the augment has a patient-specific step discontinuity.

5. The implantable device of claim 1, wherein the second surface is planar.

6. The implantable device of claim 1, wherein the second surface is piece-wise planar.

7. The implantable device of claim 1, wherein the first surface has a porous coating and is a continuously curved surface.

8. The implantable device of claim 1, wherein the first surface includes convex and concave portions.

9. The implantable device of claim 3, wherein the first surface has a step discontinuity.

10. The implantable device of claim 1, wherein the augment is a femoral augment engageable to a femoral implant of a distal femur of a knee joint of the patient.

11. The implantable device of claim 1, wherein the augment is a tibial augment engageable to a tibial implant of a distal femur of a knee joint of the patient.

12. An implantable device comprising:
an implant component for a bone of a knee joint of a patient, the knee implant component having an outer surface facing the knee joint and a second non-custom surface opposite to the outer surface; and
an augment having first and second surfaces, wherein the first surface is non-custom surface mating with the non-custom surface of the knee implant and the second surface is a three-dimensional patient specific surface that closely matches and can mate to a substantially unaltered and unresected surface of a bone defect of the specific patient only in one position.

13. The implantable device of claim 12, wherein the augment has a patient-specific and variable depth measured between the first and second surfaces of the augment.

14. The implantable device of claim 13, wherein the augment includes a peripheral surface between the first and second surfaces, and the peripheral surface is a three-dimensional patient specific surface that closely matches and can mate to a complementary surface of the bone of the specific patient.

15. The implantable device of claim 14, wherein the peripheral surface has patient-specific variable radial dimension at each depth along the augment.

16. The implantable device of claim 13, wherein the first surface of the augment is planar.

17. The implantable device of claim 12, wherein the first surface of the augment is piece-wise planar.

18. The implantable device of claim 12, wherein the implant component is a distal femoral component and the augment is a femoral augment.

19. The implantable device of claim 18, wherein the femoral augment is a femoral sleeve with a fixation bore receiving a boss of the distal femoral component in a taper fit connection.

20. The implantable device of claim 12, wherein the implant is a tibial tray and the augment is a tibial augment.

21. The implantable device of claim 12, wherein the augment is a tibial sleeve for a proximal tibial bone.

22. A method of preparing a knee joint for an implant component:
preparing a bone end of a knee joint of the patient with standard resections configured to match a non-custom bone-engaging surface of an implant component without resecting a three-dimensional surface of a defect in the bone;
attaching a first implant-engaging surface of a patient-specific augment to a portion of the bone-engaging surface of the implant;
nestingly mating a second patient-specific three-dimensional surface of the augment to the three-dimensional surface of the defect; and
attaching the implant to the bone.

23. The method of claim 22, further comprising mating a third patient-specific peripheral surface of the augment to at least a portion of a complementary surface of the bone.

24. The method of claim 22, further comprising:
reconstructing a three-dimensional image of the bone with the defect from a medical scan of the bone during a preoperative plan for the patient;
selecting the implant component for the specific patient; and
manufacturing the patient-specific augment from the three-dimensional image of the bone of the specific patient and the implant component.
